SEQUENCE LISTING (9 sequences)

SEQ ID NO: 1 - DNA, 1315 bp, Homo sapiens
agcaggacgtgagacttctacctgctcactcagaatcatttccgcaccaaccatggccacgtttgtggagctcagtacaaaagccaagatgcccattgtgggcctgggcacttggaagtctcttctcggcaaagtgaaagaagcggtgaaggtggccattgatgcagaatatcgccacattgactgtgcctatttctatgagaatcaacatgaggtgggagaagccatccaagagaagatccaagagaaggctgtgatgcgggaggacctgttcatcgtcagcaagttgtggcccactttctttgagagaccccttgtgaggaaagcctttgagaagaccctcaaggacctgaagctgagctatctggacgtctatcttattcactggccacagggattcaagactggggatgactttttccccaaagatgataaaggtaatatgatcagtggaaaaggaacgttcttggatgcctgggaggccatggaggagctggtggacgaggggctggtgaaagcccttggggtctcaaatttcaaccacttccagatcgagaggctcttgaacaaacctggactgaaatataaaccagtgactaaccaggttgagtgtcacccatacctcacgcaggagaaactgatccagtactgccactccaagggcatcaccgttacggcctacagccccctgggctctccggatagaccttgggccaaacctgaggacccttccctgctggaggatcccaagattaaggagattgctgcaaagcacaaaaaaaccacagcccaggttctgatccgtttccatatccagaggaatgtgacagtgatccccaagtctgtgacaccagcacacattgttgagaacattcaggtctttgactttaaattgagtgatgaggagatggcaaccatactcagcttcaacagaaactggagggcctttgacttcaaggaatcctctcatttggaggactttcccttcgatgcagaatattgaggttgaatctcctggtgagattacacaggggattctctttcttcgctgaagtgtgactgtctccactcaagaactattttagccaagcttatctgagatcacagtgaactttgtcctgttgtagaccagaatggaggtgctgttttagacatgtatttctgtatgttcaactaggataagaatatcacagaaaagcatggcctgaataagcaaatgacaattttttccacttatctgatctgatcaaatgtctgttaagcaccagaaactctgccaacactgaggatgtaaagataaataataaaaaataat

SEQ ID NO: 2 - protein, 316 aa, Homo sapiens
MATFVELSTKAKMPIVGLGTWKSLLGKVKEAVKVAIDAEYRHIDCAYFYENQHEVGEAIQEKIQEKAVMREDLFIVSKLWPTFFERPLVRKAFEKTLKDLKLSYLDVYLIHWPQGFKTGDDFFPKDDKGNMISGKGTFLDAWEAMEELVDEGLVKALGVSNFNHFQIERLLNKPGLKYKPVTNQVECHPYLTQEKLIQYCHSKGITVTAYSPLGSPDRPWAKPEDPSLLEDPKIKEIAAKHKKTTAQVLIRFHIQRNVTVIPKSVTPAHIVENIQVFDFKLSDEEMATILSFNRNWRAFDFKESSHLEDFPFDAEY

SEQ ID NO: 3 - protein, 316 aa, Homo sapiens
MATFVELSTKAKMPIVGLGTWKSPLGKVKEAVKVAIDAGYRHIDCAYVYQNEHEVGEAIQEKIQEKAVKREDLFIVSKLWPTFFERPLVRKAFEKTLKDLKLSYLDVYLIHWPQGFKSGDDLFPKDDKGNAIGGKATFLDAWEAMEELVDEGLVKALGVSNFSHFQIEKLLNKPGLKYKPVTNQVECHPYLTQEKLIQYCHSKGITVTAYSPLGSPDRPWAKPEDPSLLEDPKIKEIAAKHKKTAAQVLIRFHIQRNVIVIPKSVTPARIVENIQVFDFKLSDEEMATILSFNRNWRACNVLQSSHLEDYPFDAEY

SEQ ID NO: 4 - DNA, 21 bp, Artificial Sequence
  Description of Artificial Sequence Primer
atggccacgtttgtggagctc

SEQ ID NO: 5 - DNA, 21 bp, Artificial Sequence
  Description of Artificial Sequence Primer
atattctgcatcgaagggaaa

SEQ ID NO: 6 - DNA, 26 bp, Artificial Sequence
  Description of Artificial Sequence Primer
cgggatccgccacgtttgtggagctc

SEQ ID NO: 7 - DNA, 33 bp, Artificial Sequence
  Description of Artificial Sequence Primer
cccaagctttcaatattctgcatcgaagggaaa

SEQ ID NO: 8 - DNA, 22 bp, Artificial Sequence
  Description of Artificial Sequence Primer
tagaccttgggccaaacctgag

SEQ ID NO: 9 - DNA, 21 bp, Artificial Sequence
  Description of Artificial Sequence Primer
gagaggattccttgaagtcaa

(19) United States
(12) Patent Application Publication
(10) Pub. No.: US 2003/0148337 A1
(43) Pub. Date: Aug. 7, 2003

(54) ARL-2 for birth control

(75) Inventors: Monika Lessl, Glienicke-Nordbahn (DE); Michaele Peter-Kottig, Berlin (DE)

Correspondence Address: MILLEN, WHITE, ZELANO & BRANIGAN, P.C., 2200 CLARENDON BLVD., SUITE 1400, ARLINGTON, VA, 22201, US

(73) Assignee: Schering AG, Berlin, DE

(21) Appl. No.: 10/274,375

(22) Filed: Oct. 21, 2002

Related U.S. Application Data
(60) Provisional application No. 60/331,531, filed on Nov. 19, 2001.

(30) Foreign Application Priority Data
Oct. 19, 2001 (DE) 101 52 598.2

(51) Int. Cl. C12Q 1/68; C07H 21/04; C12P 21/04; C12N 9/64; C12N 5/06

(52) U.S. Cl. 435/6; 435/69.1; 435/226; 435/320.1; 435/325; 435/70.21; 536/23.2

(57) ABSTRACT

A human ARL-2 protein as well as its use for fertility disorders, therapy and diagnosis are described.

This application claims the benefit of the filing date of U.S. Provisional Application Serial No. 60/331,531 filed Nov. 19, 2001.

The invention relates to ARL-2 and its use as a pharmaceutical agent for influencing apoptotic processes, which play a basic role in birth control.

For birth control, the “standard pill” is the most frequently used means of choice as a female contraceptive agent. Its regular intake results in ovulation inhibition in women. The principle of this method is that by taking the pill, suppression of the endogenic steroid hormone production in the ovary and thus ovulation inhibition result. A drawback is that a natural cycle thus is no longer present in women. Moreover, in connection with taking the pill in patients who are potentially at risk, side effects such as, for example, tightness of the chest, weight increase, etc., can occur.

Numerous studies confirm that fertility decreases in women as they grow older. This is attributable to, i.a., a deteriorating quality of ova, an elevated abortion rate, and increased exposure to infectious germs (e.g., chlamydia or gonococci). Since, in industrial countries, however, the age of women who are pregnant for the first time is always moving further back, it is necessary to find possibilities for improving fertility. This is true for women and also for men. For couples with fertility disorders, techniques of assisted reproduction are now available (in vitro fertilization (IVF); gamete intrafallopian transfer (GIFT), intrauterine insemination). These methods are invasive, however, and are connected in most cases with a prior follicle maturation stimulation in women by proteohormones (follicle-stimulating hormone/FSH). This can result in unpleasant side effects such as headaches or pains in the abdomen, but also in the worst case in the so-called ovarian hyperstimulation syndrome.

There therefore exists the urgent need to provide new substances and agents for birth control, i.e., both to promote fertility and to inhibit fertility.

Fertility disorders are often caused by apoptotic processes and can be treated by the use of anti-apoptotic proteins. Involvement of the enzyme LHOP (Luteinizing Hormone-Dependent Ovarian Protein) that is important for fertility and ARL-1, the human homolog of LHOP, could not ensure the avoidance of apoptosis. It was not previously known, however, whether the other human homologs that have an anti-apoptotic function exist in addition to the ARL-1.

This question is answered by this invention by the preparation of a nucleic acid that comprises

a. The nucleotide sequence that is shown in Seq ID NO 1

b. A nucleotide sequence that corresponds to a sequence from a. within the scope of the degeneration of the genetic code or

c. A nucleotide sequence that hybridizes with the sequences from a. or b. under stringent conditions with the function of a human ARL-2 protein.

The term “hybridization under stringent conditions” according to this invention is defined by Sambrook et al. (Molecular Cloning, A Laboratory Manual, Cold Spring Harbor Laboratory Press, 1989). A stringent hybridization exists, for example, if after washing for 1 hour with 1×SSC and 0.1% SDS at 50° C., preferably at 55° C., especially preferably at 62° C. and most preferably at 68° C., especially for 1 hour in 0.2×SSC and 0.1% SDS at 55° C., preferably at 62° C. and most preferably at 68° C., a hybridization signal is still observed. The nucleic acids, which hybridize under these conditions with the nucleic acid that is shown in Seq. ID NO 1 or a nucleotide sequence that corresponds to this sequence within the scope of the degeneration of the genetic code, are also the subject matter of this invention.

Nucleic acids can produce single- or double-strand DNA, e.g., cDNA, or RNA, e.g., mRNA, cRNA, or pre-mRNA.

The nucleic acid that is shown in Seq ID NO 1 codes for a human ARL-2 protein.

Preferred is the nucleic acid that comprises a protein-coding section of the nucleic acid sequence that is shown in Seq ID NO 1. A protein-coding section of the sequence that is shown in Seq ID NO 1 is in the nucleotide range of 53 to 1003. ARL-2 has 93% homology to ARL-1, the human homolog of LHOP.

A subject of the invention is also a nucleic acid that codes for a polypeptide with the amino acid sequence that is shown in Seq ID NO 2.

The nucleic acids according to the invention can be obtained from mammals, e.g., human cells, or from a cDNA library or a genomic library, which is obtained from, e.g., human cells. It can be isolated according to known techniques with use of short sections of the nucleic acid sequences that are shown in Seq ID NO 1 as hybridization probes or amplification primers.

In addition, the invention relates to polypeptides that are coded by a nucleic acid according to the invention. These polypeptides have the function of a human ARL-2 protein and belong to the protein family of the aldo-keto-reductases. This group of enzymes reduces aldehyde and ketone groups of various substrates. Steroids with aldehyde or ketone groups can also belong to the substrates of the aldo-keto-reductases. The function of the ARL-2 protein is that of an aldo-keto-reductase, which is connected with apoptosis. Substrates of the ARL-2 protein, such as 4-hydroxynonenal, are cytotoxic and can induce apoptosis. It was possible to show that the aliphatic aldehyde 4-hydroxynonenal induces apoptosis in endothelial cell cultures (Herbst et al., 1997, J Cell Physiol 181, 295-303) and in neuronal cell cultures (Kruman et al., 1997, J Neurosci 17, 5089-100,; Malecki et al., 2000, J Neurochem 74, 2278-87). ARL-2 protects against apoptosis by catalyzing the reduction of these toxic and reactive aldehydes into the less reactive form of alcohol. Malfunctions in the ARL-2 protein cause the cells to undergo apoptosis. Apoptotic processes also play a very important role in the ovary, especially during the folliculogenesis. It is known that about 99% of the follicle degenerates through apoptotic processes.

In addition, a subject of the invention is a polypeptide that comprises the amino acid sequence that is shown in Seq ID NO 2.

The polypeptide according to the invention can be a recombinant polypeptide, a natural, isolated polypeptide or a synthetic polypeptide.

The polypeptide according to the invention is highly homologous to ARL-1, the human LHOP-homolog, which plays an important role in the ovulation (Brockstedt et al., 2000, Endocrinology 141, 2574-2581) and thus is of great importance for the regulation of fertility. In WO 00/52148, the sequence of the ARL-1 is described for the first time. A mouse of homologous protein was already described by Taragnat et al., 1988 (J. Reprod. Fert. 83: 835-842) in the vas deferens of male mice and referred to as MVDP (mouse vas deferens protein). The polypeptide according to the invention is distinguished by specific areas in the catalytic center, in which the amino acid sequence deviates from that of the ARL-1 protein. According to Jez et al. (Jez et al., 1997, Biochem. Pharmacol., 54, 639-647 and Jez and Penning, Chemico-Biol. Interact, 2001, 130-132, 499-525), these areas play an important role in the binding of the substrates in the catalytic center (see FIG. 1). Changes of the sequence in these areas contribute to a change in substrate specificity; ARL-2 interacts with substrates other than ARL-1 and thus represents a new enzyme, which plays an important role in the removal of apoptotic substances. ARL-2 has an anti-apoptotic action, while in the case of errors or malfunctions of ARL-2, the cells go into apoptosis.

The mRNA of the polypeptide of the invention according to Seq ID NO 2 is transcribed in various tissues, such as, for example, ovaries, adrenal glands and vas deferens.

The polypeptides according to the invention or partial areas thereof (peptides) can be used for the production of antibodies. For the production of polyclonal antibodies, the polypeptides or peptides can be bonded to, e.g., KLH (Keyhole Limpet Hemocyanin), and animals, e.g., rabbits, can be sprayed. They can also be used for the production of monoclonal antibodies. For antibody production, a polypeptide or peptide according to the invention or a mixture of several peptides according to the invention can be used. In this case, the production of the antibodies is carried out according to standard processes, as they are described in, e.g., Kohler, G. and Milstein, C., Nature 1975, 256, 495-497 and Nelson, P. N. et al., Mol. Pathol. 2000, 53, 111-117.

Subjects of the invention are also the antibodies that are directed against a polypeptide according to the invention.

The antibodies according to the invention can be used for detection of the polypeptides according to the invention. This can be carried out by, e.g., immunohistochemistry. The antibodies according to the invention can also be used in other immune tests, such as, e.g., an ELISA (enzyme linked immunosorbent assay) or in a radioimmuno test. Thus, the concentration of polypeptide according to the invention can be detected in tissue or cell extracts.

The detection of the expression of the polypeptide according to the invention can also be carried out via the detection of mRNA in the cells. The subject of the invention is therefore also the use of a probe with nucleic acid sequences that are complementary to the nucleic acid sequences that code for the peptides according to the invention for the production of a reagent for the detection of the presence of mRNA in tumor cells according to the invention. A probe is a short strand of DNA with at least 14 nucleotides. The probes according to the invention can be used in, e.g., a Northern Blot analysis. This method is described in, e.g., Sambrook, J. et al., 1989, Cold Spring Harbor Laboratory Press. Other methods for detecting RNA are in-situ hybridization, RNAse protection assay or PCR.

In addition, the invention relates to antisense molecules that are directed against the nucleic acid sequence according to the invention and can suppress the expression of the ARL-2 protein. Such molecules can be used specifically for contraception.

In addition, subjects of the invention are vectors that contain at least one copy of the nucleic acid according to the invention. Vectors can be prokaryotic or eukaryotic vectors. Examples of vectors are pPRO (Clontech), pBAD (Invitrogen), pSG5 (Stratagene), pCI (Promega), pIRES (Clontech), pBAC (Clontech), pMET (Invitrogen), pBlueBac (Invitrogen). The nucleic acids according to the invention can be inserted into these vectors with the methods that are known to one skilled in the art. In connection with expression signals, such as, e.g., promoters and enhancers, the nucleic acids according to the invention are preferably found in the vector.

The invention also relates to cells that are transfixed with a nucleic acid sequence according to the invention or with a vector according to the invention. As cells, e.g., E. coli, yeast, Pichia, Sf9, COS, CV-1 or BHK can be used. These cells can be used both for the production of the polypeptide according to the invention or for test systems.

It was possible to show that LH is secreted from the pituitary gland in the middle of the cycle and causes the meiotic maturation of the ova as well as the ovulation of the ova, and that ARL-2 is expressed specifically under control of LH. This may suggest that substances that influence (stimulate, induce or inhibit) ARL-2 regulate ova maturation, follicle maturation, steroidogenesis and/or ovulation. An inhibition of ARL-2 therefore results in an inhibition of the ova maturation, follicle maturation, steroidogenesis and/or ovulation. In addition, substances that influence (stimulate, induce or inhibit) ARL-2 also regulate the quality of the ova and follicles. In comparison to the already mentioned protein ARL-1, ARL-2 represents a new enzyme with different substrate specificity. An inhibition of the ARL-2 results in triggering apoptosis. A stimulation of the protein or an administration of the claimed recombinant protein has the opposite effect, is anti-apoptotic and promotes fertility (e.g., in the case of ovarian insufficiency or in the occurrence of polycystic ovaries).

A subject of the invention is therefore the use of the polypeptides according to the invention or the nucleic acids that code for this as a target substance for the production of a means for treating female fertility disorders, whose causes are based on a malfunction of the ARL-2 protein.

ARL-2 is expressed in the vas deferens. Substrates of the ARL-2 protein, such as the aldehyde 4-hydroxynonenal, could be identified in human ejaculate (Gomez et al., 1998, Int J Androl 21, 81-94; Selley et al;, 1991, J Reprod Fertil 92, 291-8), and elevated concentrations of 4-hydroxyalkenals are negatively correlated with seminal motility and vitality (Gomez et al., 1998, Int J Androl 21, 81-94). A malfunction of the ARL-2 protein results in a disruption in the maturation of sperm and in a reduced sperm quality and motility. A subject of the invention is therefore the use of the polypeptides according to the invention or the nucleic acids that code for this as target substances for producing an agent for treating male fertility disorders, whose cause lies in a malfunction of the ARL-2 protein.

In particular, the invention includes the use of

a. A nucleic acid according to the invention,

b. A polypeptide according to the invention, or

c. A cell according to the invention

for identifying effectors of a polypeptide according to the invention. Effectors are substances that have an inhibitory or activating effect on the polypeptide according to the invention and that are able to influence the ARL-2 function of the polypeptides according to the invention.

For the specific contraception, it may be advantageous to inhibit the ova maturation, the follicle maturation, the steroidogenesis and/or the ovulation directly. For the specific contraception, it may also be advantageous to inhibit the sperm maturation directly and/or to influence the quality of the sperm such that a fertilization of the ova can no longer take place. By administering antibodies that are directed against the polypeptide according to the invention, the endogenic function of ARL-2 can be impaired and/or eliminated. The invention therefore also relates to the use of antibodies that are directed against a polypeptide according to the invention.

In addition, the invention relates to a test system for identifying substrates and effectors of a polypeptide according to the invention, whereby a polypeptide according to the invention can be incubated as a complete or partial sequence of it with a substrate and/or modulator and, for example, the reduction of ketones or aldehydes can be measured.

The effectors of the polypeptide according to the invention can be used for treating diseases that are based on malfunctions in the ARL-1 protein. Examples of this are ovarian dysfunction, ‘autoimmune premature ovarian failure,’ inflammatory diseases and diseases of the immune system. In addition, these effectors can be used for treatment of female infertility. The effectors of the polypeptide according to the invention can also be used for treating male infertility, which are based on malfunctions in the ARL-2 protein. An example is reduced sperm quality.

The effectors of the polypeptide according to the invention can also be used for contraception in women. They block the activity of ARL-2, and it results in an inhibition of ova maturation, follicle maturation, steroidogenesis and/or ovulation. The effectors can also be used for contraception in men. They block the activity of ARL-2, they prevent the sperm maturation and/or reduce the sperm activity, so that fertilization of the ova can no longer occur.

In addition, the invention relates to a test system for identifying effectors of a polypeptide according to the invention, whereby a polypeptide according to the invention can be incubated as a whole or partial sequence of it with a modulator. An enzyme assay is thus suitable in particular for finding substances that stimulate or inhibit the activity of the polypeptide according to the invention (reduction of aldehydes and ketones). To find such substances, the ARL-2 protein is purified. For this purpose, for example, the coding area of the ARL-2 gene can be inserted into an expression vector, and the enzyme is over-expressed in procaryotic or eukaryotic cells (Ausubel et al., Current Protocols in Molecular Biology, 1997, Vol. 2; p. 16.0.1). The thus obtained recombinant protein can then be purified with known chromatographic methods, for example by size-exclusion and/or cation and/or anion exchange chromatography and/or can be purified by affinity chromatography and used after incubation in phosphate buffer with NAD(P)H as a co-factor in the enzyme test.

With the aid of the described enzyme assay, substances can be tested that stimulate or inhibit the reduction of substrates of the ARL-2 aldo-keto-reductases. The thus found stimulating substances are then used for the production of agents for promoting fertility in women and in men. The inhibiting substances are used for the production of agents that inhibit fertility in women and/or in men.

It was found that the polypeptide according to the invention produces an antigen that is absolutely necessary for ova maturation, follicle maturation, steroidogenesis and/or ovulation. The antigen could be administered directly to women who suffer from fertility disorders. A treatment with antibodies against the polypeptide according to the invention or fragments thereof can be used by women for birth control. In addition, it was found that the polypeptide according to the invention produces an antigen that is necessary for the sperm maturation and sperm quality control. A treatment with antibodies against the polypeptide according to the invention or fragments thereof could be used by men for birth control.

In addition, the invention relates to a process for the preparation of a pharmaceutical agent, whereby

a. Substances are brought into contact with a test system according to the invention,

b. The action of the substances on the test system in comparison to controls is measured,

c. A substance that in step b. shows a modulation of the activity of the polypeptides according to the invention is identified,

d. And the substance that is identified in step c. is mixed with the formulation substances that are commonly used in pharmaceutics.

The activity of the polypeptide according to the invention is defined as the aldo-keto-reductase activity of the polypeptide and the anti-apoptosis function that results from this. A substance that is identified by a process according to the invention can optionally be optimized relative to metabolic stability, activity in a test system according to the invention and/or bio-availability. To this end, methods that are common in chemistry can be used.

Suitable pharmaceutical forms for dispensing contain the active ingredient in an amount that either prevents fertility or else promotes it.

The active ingredient can be administered by itself or in combination with other active ingredients either simultaneously or sequentially. The pharmaceutical composition can contain other pharmaceutically common substances in addition to the active ingredient. The substances can be administered either orally, nasally, transdermally, via the lungs, or parenterally, for example by injection. In addition, implants, as well as vaginal rings or intrauterine systems, are conceivable.

Compositions that can be administered orally can be present in the form of tablets, capsules, powders or liquids. Compositions that can be administered by injection are usually in the form of a parenterally compatible aqueous solution or suspension. The preferred preparations consist in a form of dispensing that is suitable for oral, enteral or parenteral administration. Such forms for dispensing are, for example, tablets, film tablets, coated tablets, pills, capsules, powder or depot forms as well as suppositories. Corresponding tablets can be obtained, for example, by mixing active ingredient with known adjuvants, for example inert diluents such as dextrose, sugar, sorbitol, mannitol, polyvinylpyrrolidone, explosives such as corn starch or alginic acid, binders such as starch or gelatin, lubricants such as carboxypolymethylene, carboxymethyl cellulose, cellulose acetate phthalate or polyvinyl acetate. The tablets can also consist of several layers.

In addition, the invention relates to a process for diagnosis of diseases, whose causes include mutations of the ARL-2 protein. For this purpose, DNA chips can be used. The invention therefore relates in addition to a DNA chip, in which at least one oligonucleotide is immobilized, which corresponds to the complete cDNA sequence or a partial sequence or a complementary sequence to the one described in Seq ID 1. The invention thus also relates to the use of a DNA chip according to the invention for diagnosis of fertility disorders.

DNA chips, also known as DNA microarrays, are miniaturized vehicles, in most cases made of glass or silicon, on whose surfaces DNA molecules of known sequence are immobilized in an ordered grid in high density. The surface-bonded DNA molecules are hybridized with complementary, optionally labeled nucleic acids. The labeling can be a fluorescence dye.

In the case of oligonucleotide chips, the oligonucleotides, which can be bonded to a DNA chip according to the invention, represent partial sequences of the gene products (mRNA or cDNA that is derived therefrom). One or more oligonucleotides per gene can be bonded to the DNA chip. Preferred are 25 nucleotide-long oligonucleotides. The latter are preferably selected from the respective 3′-untranslated end of the gene. Methods for production and use of DNA chips are described in, e.g., U.S. Pat. Nos. 5,578,832; 5,556,752 and 5,510,270.

In the case of cDNA chips, the complete gene products (cDNAs) or subfragments (200-500 bp long) are bonded to the chip. The method is described in, e.g., Eckmann, L. et al., J. Biol Chem., 2000, 275 (19), 14084-14094.

First, the suitable DNA sequences are determined according to Seq ID NO 1. Sequences that can hybridize with the selected gene transcripts are suitable. The oligonucleotides are then produced on the chip by a chemical process that is based on the photolithographic process. For this purpose, photolithographic masks that were produced by suitable computer algorithms are used.

The labeled RNA is incubated with the chip in a hybridization furnace. Then, the chip is analyzed in a scanner that determines the hybridization profile. It can thus be determined whether changes to the transcript have occurred (e.g., mutations, truncations). It also makes possible the quantification of the transcript and the ARL-2 protein and sheds light on, e.g., a mutation in the promoter.

### DESCRIPTION OF THE FIGURES

FIG. 1 shows a sequence comparison of the ARL homologs 1 and 2. Deviations in the amino acid sequence of the homologs are indicated in small letters. The key amino acids that play a role in the substrate binding (Jez et al.) are identified by stars. The amino acids that play a role in the substrate binding and are different in ARL-1 and ARL-2 are identified by an arrow.

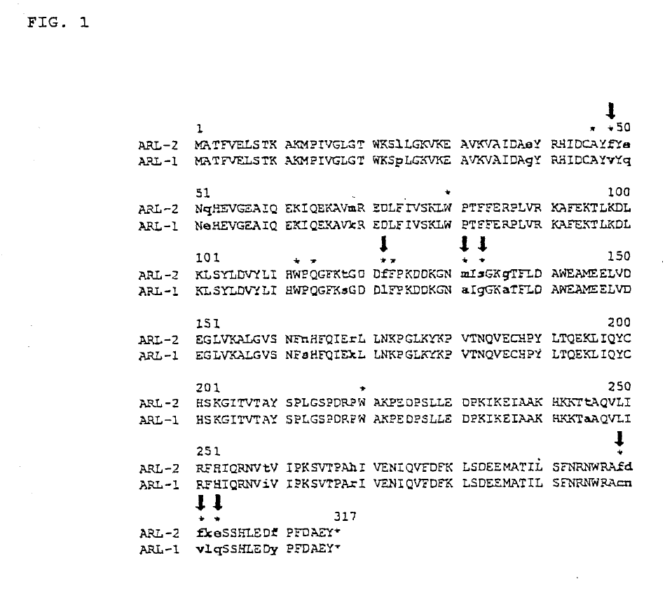

Without further elaboration, it is believed that one skilled in the art can, using the preceding description, utilize the present invention to its fullest extent. The following preferred specific embodiments are, therefore, to be construed as merely illustrative, and not limitative of the remainder of the disclosure in any way whatsoever.

In the foregoing and in the following examples, all temperatures are set forth uncorrected in degrees Celsius and, all parts and percentages are by weight, unless otherwise indicated.

### EXAMPLES

The molecular-biological methods that are used in the Examples, such as, e.g., polymerase chain reaction (PCR), production of cDNA, cloning of DNA, sequencing of DNA, were performed as described in known textbooks, such as, for example, in Molecular Cloning, A Laboratory Manual (Sambrook, J. et al., 1989, Cold Spring Harbor Laboratory Press).

### Example 1

### Cloning, Expression and Purification of Human ARL-2 Protein

For the expression of the ARL-2 protein, the coding area was amplified by means of the polymerase chain reaction (for reaction conditions, see Example 2) (primer for the N-terminal area: 5′ ATG GCC ACG TTT GTG GAG CTC 3′; primer for the C-terminal area: 5′ ATA TTC TGC ATC GAA GGG AAA 3′), and inserted in the baculovirus expression vector pBlueBac4.5/V5-His-TOPO (Invitrogen) or the eukaryotic expression vector pcDNA3.1/V5/His-TOPO (Invitrogen). For the expression of the ARL-2 protein in E. coli, the coding area was amplified by means of a polymerase chain reaction (for reaction conditions, see Example 2) (primer for the N-terminal area: 5′ CGG GAT CCG CCA CGT TTG TGG AGC TC 3′; primer for the C-terminal area: 5′ CCC AAG CTT TCA ATA TTC TGC ATC GAA GGG AAA 3′) and inserted in the procaryotic expression vector pQE30 (Quiagen). To simplify detection and purification, a fusion with an His-tag was carried out. After co-transfection of insect cells with the Bac-N-Blue DNA, recombinant viruses that were identified by a PCR process were produced. A phage stock was then applied and used in larger amounts for additional transfections and production of ARL-2. In E. coli, the expression of ARL-2 was carried out after induction by IPTG. The purification of the His-tagged proteins was carried out via a nickel affinity column according to manufacturer's information (The QIA expressionist, Qiagen). The eluted ARL-2 protein fraction was dialyzed against 10 mmol of phosphate buffer and used in an enzyme assay (see Example 3).

### Example 2

### Studies Regarding the Expression of mRNA of the Human ARL-2 Protein

To study the expression of mRNA, a PCR was carried out as follows:

As templates, cDNA from the following tissues was used: bone, vas deferens, prostate, testes, placenta, breast, ovary, adrenal gland; skin, kidney and lung. The reaction batch contains: 2 μl of cDNA; 20 μm of primer (antisense primer: 5′ TAG ACC TTG GGC CAA ACC TGA G 3′; sense primer: 5′ GAG AGG ATT CCT TGA AGT CAA 3′), 10 mmol of NTP; 1.5 mmol of MGCl₂ and 0.5 u of Taq gold (Perkin Elmer). The PCR conditions are 94° C. for 10 minutes, then 40 cycles at 94° C. for 1 minute; 58° C. for 1 minute; 72° C. for 1.5 minutes. Then, an aliquot was applied to a 1% agarose gel in 1×TAE-running buffer. Under these conditions, an expression of the mRNA in various tissues could be found (see FIG. 3).

### Example 3

### Enzyme Assay for Finding Substances that Stimulate the ARL-2 Enzyme Activity (Reduction of Aldehydes and Ketones).

A standard ARL-2 enzyme assay was performed as follows: The standard enzyme reaction batch contained 135 mmol of sodium-phosphate buffer (pH 6.3), 0.2 mmol of NADPH and measured substrate and enzyme concentrations. The enzyme reaction was carried out at 28° C., and the substrate conversion was determined by spectrometry via the reduction of A₃₄₀ of the NADPH. The reaction was started by the addition of enzyme. Controls without substrate and without enzyme were measured with respect to the standard. With the aid of the described enzyme assay, substances can be tested that stimulate the reduction of substrates of the ARL-2 protein. The aldehyde 4-hydroxynonenal and isocaproaldehyde showed themselves as preferred substrates for the aldo-keto-reductase ARL-2.

### Example 4

### Test System for Finding Substances that Inhibit the ARL-2 Enzyme Activity (Reduction of Aldehydes and Ketones).

The above-described enzyme assay should be used analogously to what is described under Example 3 to find substances that inhibit the ARL-2 activity.

### Example 5

### Test System for Finding Apoptosis-Inducing Substances

In this test system, substances can be examined with respect to their apoptosis-inducing action. The adrenocorticotrophic carcinoma cell line Y1 was used as a cell system. Cell cultures of this mouse cell line were treated for 12 hours with different substances before the rate of apoptosis in the cells was determined. 1×10⁻⁵ Y1 cells/ml were resuspended in DME/F12 (+10% FCS) and treated with the substances to be tested. After 12 hours of incubation time, the apoptosis rate was determined in the Y1 cells using annexin-V-alexa 468 staining (Roche) in combination with DNA staining (Hoechst-33258, Molecular Probes) according to manufacturer's information. Substrates of the ARL-2 protein, such as the aldehyde 4-hydroxynonenal, can induce apoptosis in the Y1 cell line at a concentration of 10 μM.

### Example 6

### Test System, in Which the Function of ARL-2 that Protects Against Apoptosis can be Studied

The above-described test system for finding apoptosis-inducing substances in the Y1 cell line (see Example 5) can be used analogously to study the anti-apoptosis function of the ARL-2 protein. Substrates of the ARL-2 protein, such as, e.g., 4-hydroxynonenal, induce apoptosis in Y1 cells. The aldo-keto reductase ARL-2 can reduce toxic aldehydes, such as, e.g., the 4-hydroxynonenal, into a less bioreactive form and as a result protect cells against apoptosis. Y1 cells were treated with the substances to be studied as described under Example 5; in addition, 12 hours before the treatment of the substance, the ARL-2 that was cloned in a eukaryotic expression vector (pCDNA 3.1, see Example 1) was transfixed in the Y1 cell by means of LipfectAMINE (Life Technology, Inc.) according to manufacturer's information. 12 hours after treatment with the substances (and consequently 24 hours after the transfection), the apoptosis rate in these cells was determined by an annexin-V staining (see Example 5). At the same time, the expression of ARL-2 was also studied by an ARL-2 staining, and the co-localization of ARL-2 and apoptosis in the cells was studied.

The entire disclosures of all applications, patents and publications, cited herein and of corresponding German Application No. 101 52 598.2 filed Oct. 19, 2001, and U.S. Provisional Application Serial No. 60/331,531 filed Nov. 19, 2001 are incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. Nucleic acid that comprises
a. The nucleotide sequence that is shown in Seq ID NO 1
b. A nucleotide sequence that corresponds to a sequence from a. within the scope of the degeneration of the genetic code or
c. A nucleotide sequence that hybridizes with the sequences from a. or b. under stringent conditions with the function of a human ARL-2 protein.

2. Nucleic acid according to claim 1 that comprises a protein-coding section of the nucleic acid sequence that is shown in Seq ID NO 1.

3. Nucleic acid that codes for a polypeptide with the amino acid sequence that is shown in Seq ID NO 2.

4. Polypeptides coded by a nucleic acid according to one of claims 1-3.

5. Polypeptide that comprises the amino acid sequence that is shown in Seq ID NO 2 or portions thereof.

6. Use of a polypeptide according to claims 4 and 5 or of portions of this polypeptide for the production of antibodies.

7. Antibodies against a polypeptide according to one of claims 4 and 5.

8. Use of a probe with nucleic acid sequences that are complementary to the nucleic acid sequences according to claims 1-3 for the production of a reagent for detecting the presence of mRNA and, according to one of claims 1-3, in cells.

9. Antisense molecule that is directed against the nucleic acid according to claim 1.

10. Vector that contains at least one copy of a nucleic acid according to one of claims 1-3.

11. Cell that is transfixed with a nucleic acid according to one of claims 1-3 or with a vector according to claim 9.

12. Use of a cell according to claim 11 for the expression of the nucleic acid according to one of claims 1-3.

13. Use of
a. A nucleic acid according to one of claims 1 to 3,
b. A polypeptide according to one of claims 4 and 5 or
c. A cell according to claim 11 for identifying effectors of a polypeptide according to claim 4 or 5.

14. Use of a nucleic acid according to claims 1 to 3 or a polypeptide according to claims 4 and 5 of the antibody according to claim 7 or an antisense molecule according to claim 9 as a target substance for the production of an agent for diseases that are causally associated with the ARL-2 gene and/or protein.

15. Test system for identifying effectors of a polypeptide according to claim 4 or 5, whereby a polypeptide according to the invention is incubated as a complete or partial sequence of it with a modulator (effector), and the amounts of reduced aldehydes and ketones are measured.

16. Test system according to claim 14 or 15, whereby the effectors inhibit or activate the aldehyde- and ketone-reducing activity.

17. Test system for identifying effectors of the enzyme activity of the polypeptide of the invention according to claim 15 in a cell of the invention, whereby the amount of reduced aldehydes and ketones is measured.

18. Test system for identifying effectors of the anti-apoptosis polypeptide function of the invention according to claim 15.

19. Process for the preparation of a pharmaceutical agent, whereby
a. Substances with a test system according to claims 15, 16 and 17 are brought into contact.
b. The action of the substances on the test system in comparison to controls is measured,
c. A substance that in step b. shows a modulation of the activity of the polypeptide according to claim 4 or 5 is identified,
d. And the substance that is identified in step c. is mixed with the formulation substances that are commonly used in pharmaceutics.

20. DNA chip, characterized in that at least one oligonucleotide is immobilized, which corresponds to the complete cDNA sequence or a partial sequence or a complementary sequence to the one described in Seq ID NO 1.

21. Use of a DNA chip according to claim 20 for diagnosis of fertility disorders.
